(19) United States
(12) Patent Application Publication
(10) Pub. No.: US 2024/0327795 A1
(43) Pub. Date: Oct. 3, 2024

(54) METHODS OF GENERATING MESENCHYAL STEM CELLS WHICH SECRETE NEUROTROPHIC FACTORS IN A BIOREACTOR SYSTEM

(71) Applicant: BRAINSTORM CELL THERAPEUTICS LTD., Petach Tikva (IL)

(72) Inventors: Yossef LEVY, Modi'in (IL); Jonathan SEMO, Giv'atayim (IL); Lena ELSTER KHOMSKY, Tel Aviv (IL); Keren MIZRAHI, Tel Aviv (IL)

(73) Assignee: BRAINSTORM CELL THERAPEUTICS LTD., Petach Tikva (IL)

(21) Appl. No.: 18/576,102

(22) PCT Filed: Jul. 5, 2022
(86) PCT No.: PCT/IL2022/050718

§ 371 Date: Jan. 3, 2024

Related U.S. Application Data
(60) Provisional application No. 63/218,541, filed on Jul. 6, 2021.

(51) Int. Cl. C12N 5/0775; C12M 1/12

(52) U.S. Cl. CPC C12N 5/0663; C12M 25/10; C12N 2500/32; C12N 2500/38

(57) ABSTRACT

The disclosure relates to a method for generating cells which secrete neurotrophic factors (NTFs) in a functionally closed and automated hollow-fiber bioreactor system comprising inducing differentiation of a population of undifferentiated mesenchymal stem cells (MSCs) in a differentiating medium supplemented with ascorbic acid. The disclosure further relates to a method of treating a disease for which administration of neurotrophic factor is beneficial and to a pharmaceutical composition including the described cells.

### BACKGROUND OF THE INVENTION

NurOwn® (mesenchymal stem cells-secreting neurotrophic factors MSC-NTF cells) is a cell therapy originating from a patient's own mesenchymal stem cells (MSCs). MSCs are non-hematopoietic stem cells with potential to differentiate into several cell types, (such as into the adipogenic, osteogenic and chondrogenic lineages). MSCs have broad immunomodulatory effects and ability to promote repair by direct cell replacement in some tissues or indirectly by secreting numerous trophic factors. MSCs can be isolated from, for example, bone marrow, adipose tissue, umbilical cord blood, Wharton jelly, peripheral blood and dental pulp, among others. MSCs are isolated from the bone marrow, propagated ex-vivo and induced to differentiate into cells that produce large amounts of neurotrophic factors, which are proteins that help nerve cells grow and survive.

Autologous bone marrow (BM)-MSCs are isolated from patients, expanded, cryopreserved, and, in advance of each treatment cycle, the intermediate MSCs products are thawed and induced to differentiate for the manufacturing of repeat doses of MSC-NTF cells.

The current methods used for clinical manufacturing of the intermediate MSCs product in two Chamber CellStacks (tissue culture vessels) comprises multiple open processing steps performed in a Grade A biosafety cabinet (BSC) located in a Grade B clean room. In the course of production, the clean room and the BSC are monitored on a regular basis for viable and non-viable particles, to ensure that the air handling system, cleaning procedures and personnel activities maintain regulatory quality standards. For widespread commercial application, the manufacturing process must be cost effective, safe and reproducible. The adherent nature of the MSCs requires a large surface area and multiple culture flasks for cell expansion, resulting in significant time and labor-intensive procedures with possible inter-flask heterogeneity and increased risk of microbial contamination.

Therefore, there is still a need to develop a method that would replace the current manufacturing process method in CellStacks with an automated and closed bioreactor system that is able to support the propagation of a large numbers of cells.

### SUMMARY OF THE INVENTION

In some embodiments, the present disclosure relates to a method for generating cells which secrete neurotrophic factors (NTFs) in a functionally closed and automated hollow-fiber bioreactor system comprising inducing differentiation of a population of undifferentiated mesenchymal stem cells (MSCs) in a differentiating medium supplemented with ascorbic acid.

In one embodiment, the yield of the cells is improved in comparison to generation of the cells in a differentiation medium without ascorbic acid.

In one embodiment, the ascorbic acid concentration in the differentiating medium is 250 μM.

In one embodiment, the method further comprises culturing the population of undifferentiated mesenchymal stem cells (MSCs) prior to the inducing of differentiation, wherein the culturing is affected under conditions that do not promote cell differentiation.

In one embodiment, the population of undifferentiated mesenchymal stem cells (MSCs) is cultured in a functionally closed and automated hollow-fiber bioreactor system. In one embodiment, the method comprises
a. Seeding between 10-20×10⁶ MSCs in growth media (PM) for six or seven days;
b. Propagating MSCs by using a feeding program based on lactate level measurements.
c. Replacing the PM with the differentiating media (S2M) six or seven days after seeding;
d. Incubating the cultures for three additional days;
e. Harvesting the MSC-NTF cells.

In one embodiment, the method further comprises analyzing the expression of CD73, CD90 and CD105 surface markers. In one embodiment, the method further comprises analyzing apoptosis. In one embodiment, the method further comprises analyzing VEGF specific productivity.

In some embodiments, the present disclosure relates to an isolated population of cells, which secrete neurotrophic factors, generated according to the method described above.

In one embodiment, the isolated population express mesenchymal stem cells surface markers comprising CD73, CD90 and CD105, as detected by flow cytometry.

In one embodiment, the isolated population does not express surface markers comprising CD14, CD34, CD45 and HLA-DR. In another embodiment, the isolated population does not express surface markers comprising CD3, CD19, CD14, CD34, CD45 and HLA-DR.

In one embodiment, the isolated population of cells secrete not less than 7000 pg VEGF/10⁶ cells.

In some embodiments, the present disclosure relates to a method of treating a disease for which administration of neurotrophic factors is beneficial in a subject in need thereof, comprising administering to the subject a therapeutically effective amount of the isolated population of cells as described above, thereby treating said disease.

In one embodiment, the disease is a neurodegenerative disease or an immune disease. In one embodiment, the neurodegenerative disease is selected from a group comprising Parkinson's, Multiple System Atrophy (MSA), multiple sclerosis, epilepsy, amyotrophic lateral sclerosis (ALS), stroke, autoimmune encephalomyelitis, diabetic neuropathy, glaucomatous neuropathy, Alzheimer's disease, and Huntington's disease. In another embodiment, the neurodegenerative disease is ALS.

In one embodiment, the immune disease is an autoimmune disease. In one embodiment, the autoimmune disease is myasthenia gravis.

In one embodiment, the administration is intramuscularly or intrathecally. In another embodiment, the administration is intramuscularly. In another embodiment, the administration is intrathecally.

In some embodiments, the present disclosure relates to a pharmaceutical composition comprising the isolated population of cells as described above as an active agent and a pharmaceutically acceptable carrier.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 is a flowchart describing the production of the MSC-NTF cells in the Quantum bioreactor system. Mononuclear cells (MNC) were separated from total bone marrow either A. manually by density gradient centrifugation in a Ficoll tube (D47, D48 and D49) or B. automatically using a Sepax2 device (D54, D55 D56, and D59). C. Isolated MNC derived from the manual or from the automatic procedures, were loaded into the Quantum bioreactor systems for two passages (P0 and P1) for enrichment and propagation of MSCs. D. MSCs were cryopreserved and E. thawed F. Propagation and differentiation of MSCs into MSC-NTF cells. G. Harvesting MSC-NTF cells, packaging and labeling “NurOwn” final product for transplantation;

FIGS. 2A-2B are an illustration of lactate generation level in the Quantum system. The level of Lactate was monitored during differentiation of MSC to MSC-NTF cells in the Quantum system control (FIG. 2A) and in the Quantum system with Ascorbic Acid (FIG. 2B). Lactate measurements were taken at the same time point for both Quantum systems;

FIGS. 3A-3B are an illustration of glucose consumption rate. The level of Glucose was monitored during differentiation of MSC to MSC-NTF cells in the Quantum system control (FIG. 3A) and in the Quantum system with Ascorbic Acid (FIG. 3B). Glucose measurements were taken at the same time points for both Quantum systems;

FIGS. 4A-4B are an illustration of apoptosis of D49 MSC-NTF cells induced to differentiate in the Quantum Control (FIG. 4A) and Quantum with Ascorbic acid (FIG. 4B) systems. Histograms were generated using CytExpert software, showing the expression of Annexin V labeled with FITC. Annexin V positive cells are shown in black;

FIG. 5 is an illustration of MSC-NTF cells' VEGF Specific productivity. Assays were performed in technical triplicates and VEGF specific productivity (VEGF secretion per 1×10⁶ cells) was calculated for MSC-NTF cells derived from the Quantum system with or without the presence of Ascorbic acid. Quantum system samples, which were taken from the sample port by syringe, were diluted by 1:20. VEGF specific productivity was also calculated for MSC-NTF cells derived from the Quantum system by taking a sample from the waste bag (1-3 liter) on the day of harvesting;

FIG. 6 is an illustration of quantitative real time PCR analysis. Expression of selected genes (BMP2, PCSK1, HGF and TOP2A) in MSC and MSC-NTF cells derived from the Quantum systems were determined by qRT-PCR analysis. Gene expression was normalized to B2M and EF1A and each result represents the average of triplicates. Differences between groups were determined by Student t test (Statistical significance is determined as p<0.05);

FIGS. 7A-7B are an illustration of immunomodulatory properties of MSC-NTF cells by inhibition of PBMC proliferation and by suppression of TNF-α and IFN-γ secretion. MSC-NTF cells derived from the Quantum system with or without Ascorbic acid were co-cultured with CFSE-labeled, PHA activated PBMC. After 4 days, the percentage of proliferating CD4 and CD8 T cells was measured by flow cytometry and was calculated as a percentage of the activated PBMC cells (FIG. 7A). The percentage of inhibition of CD4 and CD8 T cells in the presence of MSC-NTF cells is shown. Cytokine secretion was measured in the supernatants and inhibition was calculated as a percent of activated PBMC control (FIG. 7B);

FIGS. 8A-8B are an illustration of MSC-NTF cells' induction of neurite growth. Human neuroblastoma cell line SH-SY5Y neurons were co-cultured with MSC-NTF cells for 4 days (using a transwell system). Cells were imaged using the Incucyte S3 live imaging system, and neurite length was calculated using the Neurotrack module. Each sample was seeded in triplicates in media without serum. “With serum” and “Without serum” samples that did not contain cells, are shown in black full and dotted lines, respectively. Neurite growth in the presence of D48 and D49 MSC-NTF cells derived from Quantum and Quantum with Ascorbic acid (FIG. 8A). Neurite growth in the presence of D54, D55 and D59 MSC-NTF cells derived from Quantum and Quantum with Ascorbic acid (FIG. 8B).

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

### Method for Generating Cells

In some embodiments, the present disclosure relates to a method for generating cells which secrete neurotrophic factors (NTFs) in a functionally closed and automated hollow-fiber bioreactor system comprising inducing differentiation of a population of undifferentiated mesenchymal stem cells (MSCs) in a differentiating medium supplemented with ascorbic acid. In one embodiment, the yield of the cells is improved in comparison to generation of the cells in a differentiation medium without ascorbic acid.

A skilled artisan would appreciate the term “mesenchymal stem cells” or “MSCs” as adult cells which are not terminally differentiated, which can divide to yield cells that are either stem cells, or which, irreversibly differentiate to give rise to cells of a mesenchymal (chondrocyte, osteocyte and adipocyte) cell lineage. The mesenchymal stem cells of the present disclosure, in at least some embodiments, may be of an autologous or allogeneic source. Mesenchymal stem cells may be isolated from various tissues including but not limited to bone marrow, peripheral blood, cord blood, placenta Wharton jelly and adipose tissue.

A skilled artisan would appreciate the term “neurotrophic factor” or “NTF” as a cell-secreted factor that acts on the central nervous system comprising growth, differentiation, functional maintenance and/or survival effects on neurons. Examples of neurotrophic factors include, but are not limited to, glial derived neurotrophic factor (GDNF), brain-derived neurotrophic factor (BDNF), Vascular Endothelial Growth Factor (VEGF), Hepatocyte Growth Factor (HGF), Granulocyte Stimulating factor (G-CSF), Leukemia inhibitory factor (LIF), Tumor necrosis factor-inducible gene 6 protein (TSG-6), a Neurotrophin-4, insulin growth factor-I (IGF-1), Growth and differentiation Factor (GDF-15), Granulocyte Stimulating factor (G-CSF), a Tumor necrosis factor-inducible gene 6 protein (TSG-6; also known as TNF-stimulated gene 6 protein), Bone morphogenetic protein 2 (BMP2), Fibroblast Growth Factor 2 (FGF2), neurotrophin-3 (NT-3); neurotrophin-4/5; Neurturin (NTN), Neurotrophin-4, GenBank Accession No. M86528; Persephin; artemin (ART), ciliary neurotrophic factor (CNTF), and Neublastin.

In one embodiment, the ascorbic acid concentration in the differentiating medium is between 5-500 μM. In another embodiment, the ascorbic acid concentration in the differentiating medium is 5 μM. In another embodiment, the ascorbic acid concentration in the differentiating medium is 10 μM. In another embodiment, the ascorbic acid concentration in the differentiating medium is 50 μM. In another embodiment, the ascorbic acid concentration in the differentiating medium is 80 μM. In another embodiment, the ascorbic acid concentration in the differentiating medium is 100 μM. In another embodiment, the ascorbic acid concentration in the differentiating medium is 200 μM. In another embodiment, the ascorbic acid concentration in the differentiating medium is 250 μM. In another embodiment, the ascorbic acid concentration in the differentiating medium is 300 μM. In another embodiment, the ascorbic acid concentration in the differentiating medium is 400 μM. In another embodiment, the ascorbic acid concentration in the differentiating medium is 500 μM.

The current disclosure discloses a series of experiments demonstrating that the presence of ascorbic acid in the differentiation serum free medium is essential to produce at least one MSC-NTF cells' product dose in the Quantum system. In one embodiment, a product dose comprises between 100-125×10⁶ cells. In another embodiment, a product dose comprises 100×10⁶ cells. In another embodiment, a product dose comprises 105×10⁶ cells. In another embodiment, a product dose comprises 110×10⁶ cells. In another embodiment, a product dose comprises 115×10⁶ cells. In another embodiment, a product dose comprises 120×10 cells. In another embodiment, a product dose comprises 125×10⁶ cells.

In one embodiment, the method does not involve any genetic manipulation.

In one embodiment, the method further comprises culturing the population of MSCs prior to inducing differentiation, wherein the culturing is affected under conditions that do not promote cell differentiation.

In one embodiment, the population of undifferentiated MSCs is cultured, prior to inducing differentiation, in a functionally closed and automated hollow-fiber bioreactor system.

In one embodiment, the population of undifferentiated MSCs is cultured for a duration based on lactate parameters. In one embodiment, the duration is determined by lactate levels above 4 mmol/L. In another embodiment, the duration is determined by lactate levels above 5 mmol/L. In another embodiment, the duration is determined by lactate levels above 6 mmol/L. In another embodiment, the duration is determined by lactate levels above 7 mmol/L. In another embodiment, the duration is determined by lactate levels above 8 mmol/L. In another embodiment, the duration is determined by lactate levels above 9 mmol/L.

In one embodiment, the undifferentiated MSCs are being cryopreserved, prior to induction of the differentiation process.

In one embodiment, the differentiating medium is designated S2M media. In one embodiment, the S2M media comprises DMEM, L-Glutamine, Sodium Pyruvate, di-butyryl cyclic AMP (dbcAMP), human Basic Fibroblast Growth Factor (bFGF), human platelet derived growth factor (PDGF-AA) and human Heregulinβ. In another embodiment, the differentiation medium is serum free.

In one embodiment, the growth medium is PM media. In one embodiment, the PM media is based on human Platelet lysate. In another embodiment, the PM media comprises 10% platelet lysate. In one embodiment, a continuous supply of the growth medium (PM) is supplied from a PM medium bag. In one embodiment, the flow rate is 0.1 ml/min. In another embodiment, the flow rate is 0.8-1.6 ml/min.

In one embodiment, the method further comprises M2 media. In one embodiment, the M2 media comprises DMEM, L-Glutamine and Sodium Pyruvate. In another embodiment, the M2 media is supplemented with Ascorbic Acid.

In one embodiment, the method comprises
a. Seeding between 10-20×10⁶ MSC in growth media (PM) for six or seven days;
b. Propagating MSC by using a feeding program based on lactate level measurements;
c. Replacing the PM with the differentiating media (S2M) six or seven days after seeding;
d. Incubating the cultures for three additional days;
e. Harvesting the MSC-NTF cells.

In some embodiments, the method further comprises analyzing the expression of CD73, CD90 and CD105 surface markers. In one embodiment, the method further comprises analyzing the expression of CD73 surface marker. In another embodiment, the method further comprises analyzing the expression of CD90 surface marker. In another embodiment, the method further comprises analyzing the expression of CD105 surface marker.

Analyzing cell surface markers may be performed by using any method known in the art including for example, flow cytometry, High Performance Liquid chromatography (HPLC), immunohistochemistry or in situ-PCR. In one embodiment, the analysis of the expression of surface markers is performed by Flow cytometry. In another embodiment, the analysis of the expression of surface markers is performed by High Performance Liquid chromatography (HPLC). In another embodiment, the analysis of the expression of surface markers is performed by immunohistochemistry. In another embodiment, the analysis of the expression of surface markers is performed by in situ-PCR.

A skilled artisan would appreciate the term “flow cytometry” as an assay in which the proportion of a material (e.g. blood cells comprising a particular marker) in a sample is determined by labeling the material (e.g., by binding a labeled antibody to the material), causing a fluid stream containing the material to pass through a beam of light, separating the light emitted from the sample into constituent wavelengths by a series of filters and mirrors, and detecting the light. A multitude of flow cytometers are commercially available including for e.g. Becton Dickinson FACScan and FACScalibur (BD Biosciences, Mountain View, CA), the Beckman Coulter Life Sciences Cytomics FC 500 series and the CytoFLEX, CytoFLEX S and CytoFLEX LX series (Beckman Coulter Life Sciences, Indianapolis, IN). Antibodies that may be used for FACS analysis are taught in Schlossman S, Boumell L, et al, [Leucocyte Typing V. New York: Oxford University Press; 1995] and are widely commercially available.

In some embodiments, the method further comprises analyzing apoptosis. Analyzing apoptosis may be performed by using any method known in the art. In one embodiment, the analysis of apoptosis is performed by an Annexin V Apoptosis Detection Kit FITC.

In some embodiments, the method further comprises analyzing Vascular endothelial growth factor (VEGF) specific productivity. The amount of VEGF can be quantified using an VEGF ELISA assay (VEGF DuoSet R&D systems, Cat: DY293B) for example and without limitation.

### Isolated Population of Cells

In some embodiments, the present disclosure relates to an isolated population of cells, which secrete neurotrophic factors, generated according to the method described above.

In one embodiment, the isolated population express mesenchymal stem cells surface markers comprising CD73, CD90 and CD105. In another embodiment, the isolated population express mesenchymal stem cells surface marker CD73. In another embodiment, the isolated population express mesenchymal stem cells surface marker CD90. In another embodiment, the isolated population express mesenchymal stem cells surface marker CD105. In one embodiment, the surface markers comprising CD73, CD90 and CD105 are detected by flow cytometry.

In one embodiment, the isolated population does not express surface markers comprising CD14, CD34, CD45 and HLA-DR. In another embodiment, the isolated population does not express surface markers comprising CD3, CD14, CD19, CD34, CD45 and HLA-DR. In another embodiment, the isolated population does not express surface marker CD3. In another embodiment, the isolated population does not express surface marker CD14. In another embodiment, the isolated population does not express surface marker CD19. In another embodiment, the isolated population does not express surface marker CD34. In another embodiment, the isolated population does not express surface marker CD45. In another embodiment, the isolated population does not express surface marker HLA-DR.

In one embodiment, the isolated population of cells secrete not less than 7000 pg VEGF/10⁶ cells. In another embodiment, the isolated population of cells secrete not less than 10,000 pg VEGF/10⁶ cells. In another embodiment, the isolated population of cells secrete not less than 20,000 pg VEGF/10⁶ cells. In another embodiment, the isolated population of cells secrete not less than 30,000 pg VEGF/10⁶ cells. In another embodiment, the isolated population of cells secrete not less than 40,000 pg VEGF/10⁶ cells. In another embodiment, the isolated population of cells secrete not less than 50,000 pg VEGF/10⁶ cells. In another embodiment, the isolated population of cells secrete not less than 60,000 pg VEGF/10⁶ cells. In another embodiment, the isolated population of cells secrete not less than 70,000 pg VEGF/10⁶ cells. In another embodiment, the isolated population of cells secrete not less than 80,000 pg VEGF/10⁶ cells. In another embodiment, the isolated population of cells secrete not less than 90,000 pg VEGF/10⁶ cells. In another embodiment, the isolated population of cells secrete not less than 100,000 pg VEGF/10⁶ cells. In another embodiment, the isolated population of cells secrete not less than 110,000 pg VEGF/10⁶ cells.

### Method of Treating a Disease.

In some embodiments, the present disclosure relates to method of treating a disease for which administration of neurotrophic factors is beneficial in a subject in need thereof, comprising administering to the subject a therapeutically effective amount of the isolated population of cells as described above, thereby treating said disease.

The term “therapeutically effective amount” refers in one embodiment, to an amount of the isolated population of cells as described above sufficient to elicit a protective immune response in the subject to which it is administered. The immune response may comprise, without limitation, induction of cellular immunity.

In one embodiment, the cells are ex vivo differentiated from MSCs which are allogeneic to said subject. In another embodiment, the cells are ex vivo differentiated from autologous MSCs which are derived from the bone marrow of said subject.

In one embodiment, the disease is a neurodegenerative disease or an immune disease. In one embodiment, the neurodegenerative disease is selected from the group comprising amyotrophic lateral sclerosis (ALS), Parkinson's, Multiple System Atrophy (MSA), multiple sclerosis, epilepsy, stroke, autoimmune encephalomyelitis, diabetic neuropathy, glaucomatous neuropathy, Alzheimer's disease, and Huntington's disease. In another embodiment, the neurodegenerative disease is amyotrophic lateral sclerosis (ALS).

In another embodiment, the neurodegenerative disease is Parkinson's. In another embodiment, the neurodegenerative disease is Multiple System Atrophy (MSA). In another embodiment, the neurodegenerative disease is multiple sclerosis. In another embodiment, the neurodegenerative disease is epilepsy. In another embodiment, the neurodegenerative disease is stroke. In another embodiment, the neurodegenerative disease is autoimmune encephalomyelitis. In another embodiment, the neurodegenerative disease is diabetic neuropathy. In another embodiment, the neurodegenerative disease is Alzheimer's disease. In another embodiment, the neurodegenerative disease is Huntington's disease.

In one embodiment, the immune disease is an autoimmune disease. In one embodiment, the autoimmune disease is selected from myasthenia gravis, neuromyelitis optica spectrum disorder (NMOSD), optic neuritis (ON), transverse myelitis (TM) and Systemic lupus erythematosus (SLE). In another embodiment, the autoimmune disease is myasthenia gravis. In another embodiment, the autoimmune disease is neuromyelitis optica spectrum disorder (NMOSD). In another embodiment, the autoimmune disease is optic neuritis (ON). In another embodiment, the autoimmune disease is transverse myelitis (TM). In another embodiment, the autoimmune disease is Systemic lupus erythematosus (SLE).

In one embodiment, the administration is intramuscularly or intrathecally. In another embodiment, the administration is intramuscularly and intrathecally. In another embodiment, the administration is intramuscularly. In another embodiment, the administration is intrathecally.

In one embodiment, when the administering is intramuscularly, a total amount of MSC-NTF cells administered to a subject is between 20-100×10⁶ cells per administration. In another embodiment, when the administering is intramuscularly, a total amount of MSC-NTF cells administered to a subject is 20×10⁶ cells per administration. In another embodiment, when the administering is intramuscularly, a total amount of MSC-NTF cells administered to a subject is 40×10⁶ cells per administration. In another embodiment, when the administering is intramuscularly, a total amount of MSC-NTF cells administered to a subject is 60×10⁶ cells per administration. In another embodiment, when the administering is intramuscularly, a total amount of MSC-NTF cells administered to a subject is 80×10⁶ cells per administration. In another embodiment, when the administering is intramuscularly, a total amount of MSC-NTF cells administered to a subject is 100×10⁶ cells per administration.

In one embodiment, when the administering is intrathecally, a total amount of MSC-NTF cells administered to a subject is between 50-200×10⁶ cells per administration. In another embodiment, a total amount of MSC-NTF cells administered to a subject is between 100-125×10⁶ cells per administration. In another embodiment, when the administering is intrathecally, a total amount of MSC-NTF cells administered to a subject is 50×10⁶ cells per administration. In another embodiment, when the administering is intrathecally, a total amount of MSC-NTF cells administered to a subject is 100×10⁶ cells per administration. In another embodiment, when the administering is intrathecally, a total amount of MSC-NTF cells administered to a subject is 125×10⁶ cells per administration. In another embodiment, when the administering is intrathecally, a total amount of MSC-NTF cells administered to a subject is 150×10⁶ cells per administration. In another embodiment, when the administering is intrathecally, a total amount of MSC-NTF cells administered to a subject is 200×10⁶ cells per administration.

In one embodiment, when the administering is intrathecally and intramuscularly, a total amount of MSC-NTFs administered to a subject is between 20-500×10⁶ cells. In another embodiment, when the administering is intrathecally and intramuscularly, a total amount of MSC-NTFs administered to a subject is 20×10⁶ cells. In another embodiment, when the administering is intrathecally and intramuscularly, a total amount of MSC-NTFs administered to a subject is 50×10⁶ cells. In another embodiment, when the administering is intrathecally and intramuscularly, a total amount of MSC-NTFs administered to a subject is 100×10⁶ cells. In another embodiment, when the administering is intrathecally and intramuscularly, a total amount of MSC-NTFs administered to a subject is 150×10⁶ cells. In another embodiment, when the administering is intrathecally and intramuscularly, a total amount of MSC-NTFs administered to a subject is 200×10⁶ cells. In another embodiment, when the administering is intrathecally and intramuscularly, a total amount of MSC-NTFs administered to a subject is 250×10⁶ cells. In another embodiment, when the administering is intrathecally and intramuscularly, a total amount of MSC-NTFs administered to a subject is 300×10⁶ cells. In another embodiment, when the administering is intrathecally and intramuscularly, a total amount of MSC-NTFs administered to a subject is 350×10⁶ cells. In another embodiment, when the administering is intrathecally and intramuscularly, a total amount of MSC-NTFs administered to a subject is 400×10⁶ cells. In another embodiment, when the administering is intrathecally and intramuscularly, a total amount of MSC-NTFs administered to a subject is 450×10⁶ cells. In another embodiment, when the administering is intrathecally and intramuscularly, a total amount of MSC-NTFs administered to a subject is 500×10⁶ cells.

In one embodiment, when the administering is intramuscularly, the number of administrations per muscle may vary from 5-50, 10-30, 20-100, or from 15-25 during the course of the treatment.

Depending on the severity and responsiveness of the condition to be treated, dosing of cells can be of a single or a plurality of administrations, with course of treatment lasting from several days to several weeks or months depending on when diminution of the disease state or slowing the disease progression are achieved.

In one embodiment, the administration is a single administration. In another embodiment, the administration is a repeated administration.

In one embodiment, the repeated administration is up to 10 administrations. In another embodiment, the repeated administration is 2 administrations. In another embodiment, the repeated administration is 3 administrations. In another embodiment, the repeated administration is 4 administrations. In another embodiment, the repeated administration is 5 administrations. In another embodiment, the repeated administration is 6 administrations. In another embodiment, the repeated administration is 7 administrations. In another embodiment, the repeated administration is 8 administrations. In another embodiment, the repeated administration is 9 administrations. In another embodiment, the repeated administration is 10 administrations.

In one embodiment, the repeated administration is once every 4 weeks. In another embodiment, the repeated administration is once every 5 weeks. In another embodiment, the repeated administration is once every 6 weeks. In another embodiment, the repeated administration is once every 7 weeks. In another embodiment, the repeated administration is once every 8 weeks. In another embodiment, the repeated administration is once every 9 weeks. In another embodiment, the repeated administration is once every 10 weeks. In another embodiment, the repeated administration is once every 11 weeks. In another embodiment, the repeated administration is once every 12 weeks.

In one embodiment, the repeated administration is from a single bone marrow aspirate.

The cells of the present invention, in at least some embodiments, may be co administered with therapeutic agents useful in treating neurodegenerative disorders, such as gangliosides; antibiotics, neurotransmitters, neurohormones, toxins, neurite promoting molecules; and antimetabolites small molecule agents and precursors of neurotransmitter molecules such as L-DOPA. For ALS, for example the cells of the present invention may be co-administered with Rilutek® (riluzole, Sanofi Aventis), or Edaravone (Radicava) Additionally, or alternatively, the cells of the present invention, in at least some embodiments, may be co-administered with other cells capable of synthesizing a neurotransmitter.

In some embodiments, the present disclosure relates to a pharmaceutical composition comprising the isolated population of cells as described above as an active agent and a pharmaceutically acceptable carrier.

A skilled artisan would appreciate the term “pharmaceutical composition” as a preparation of the cell population described herein, with other chemical components such as pharmaceutically suitable carriers and excipients. The purpose of a pharmaceutical composition is to facilitate administration of a compound to a subject.

A skilled artisan would appreciate the term “pharmaceutically acceptable carrier” as a carrier or a diluent that does not cause significant irritation to a subject and does not abrogate the biological activity and properties of the administered compound. Examples, without limitations, of carriers are Plasmalyte, saline; buffers; culture medium such as DMEM or RPMI; hypothermic storage medium containing components that scavenge free radicals, provide pH buffering, oneotic/osmotic support, energy substrates and ionic concentrations that balance the intracellular state at low temperatures; and mixtures of organic solvents with water.

A skilled artisan would appreciate the term “excipient” as an inert substance added to a pharmaceutical composition to further facilitate administration of a compound and maintain cells viability at a pre-determined temperature for a suitable period of time before transplantation/injection. Examples, without limitation, of excipients include albumin, plasma, serum and cerebrospinal fluid (CSF), antioxidants such as NAcetylcysteine (NAC) or resveratrol.

The amount of a composition to be administered will be dependent on the individual being treated, the severity of the affliction, the manner of administration, the judgment of the prescribing physician, etc. The dosage and timing of administration will be responsive to a careful and continuous monitoring of the individual changing condition. For example, a treated ALS patient will be administered with an amount of cells which is sufficient to alleviate the symptoms of the disease, based on the monitoring indications.

In one embodiment, the cell population is provided in a ready-to-use treatment package with the appropriate primary and secondary labels. In one embodiment, the treatment package consists of one 5 mL syringe for Intrathecal (IT) administration. Each treatment package consists of a ready-for-injection syringe containing MSC-NTF cells at a dose of 100-125×10⁶ cells in 4 ml.

In one embodiment, syringes are capped with a stopper (not a needle). The 5 mL syringe for IT administration is packed in a pouch.

In one embodiment, the treatment package is delivered to the Medical Center in a shipping system container designed for maintaining a temperature of 2-8° C. during shipment. In one embodiment, the product can be administered to the patient within the established shelf life of the product.

In another embodiment, the treatment package consists of one Cryotube containing 130×10⁶ MSC-NTF cells/tube for IT administration. The Cryotubes are shipped in the liquid nitrogen vapor phase and the tube is thawed by the patient's bed.

### EXAMPLES

### Example 1-MSC-NTF Cells' Differentiation in Bioreactor Supplemented with Ascorbic Acid

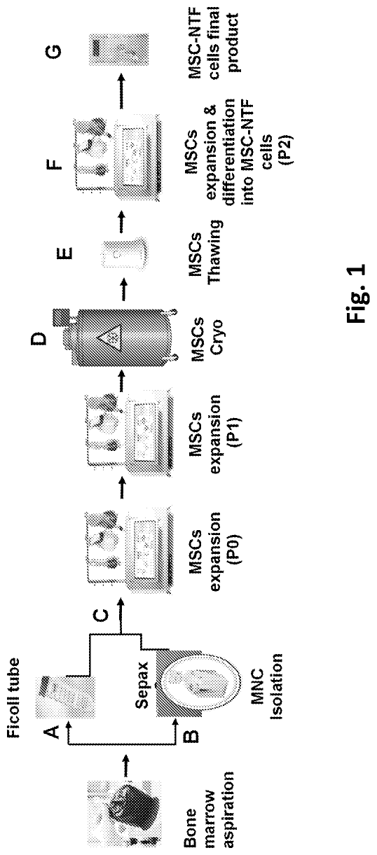

In this study, Mononuclear cells (MNC) derived from fresh BM aspirates of healthy donors were isolated manually by density gradient centrifugation (D48, D49) or automatically using the Sepax2 system (D54, D55 and D59). No significant difference was found between the two MNC separation methods (p=0.52, data not shown). MNC isolated from each BM sample were loaded into the Quantum system (FIG. 1). MSCs in the Quantum bioreactor system were harvested and cryopreserved (Table 1).

TABLE 1

| Donor | MNC Isolation | MSC Culture | MSC-NTF cells |
|---|---|---|---|
| ID | Method | system | Culture System |
| D48 | Ficoll ®-Paque | Quantum ® | Quantum (Control) |
| | PREMIUM | | Quantum with AA |
| D49 | Ficoll ®-Paque | Quantum ® | Quantum (Control) |
| | PREMIUM | | Quantum with AA |
| D54* | Sepax 2 | Quantum ® | Quantum (Control) |
| | | | Quantum with AA |
| D55 | Sepax 2 | Quantum ® | Quantum (Control) |
| | | | Quantum with AA |
| D59 | Sepax 2 | Quantum ® | Quantum (Control) |
| | | | Quantum with AA |

*In D54 run (one out of five donors) 10 × 10⁶ thawed MSCs were seeded.
MSC-NTF cells' differentiation in the Quantum System was induced as described below.

### Seeding MSC

P1 MSCs that were propagated in a Quantum system and cryopreserved were thawed into PM medium, and 20×10⁶ MSCs (in 100 ml PM growth media) were loaded into each one of the two Quantum system cell inlet bags (Table 1). Cells were loaded onto the Intracapillary (IC) side of the bioreactor utilizing the ‘Load Cells with Circulation’ task. During this task, the bioreactor was in the “in motion” mode rotating from −180° to 270°. This task was comprised of a series of 6 steps: Steps 1, 3 and 5:7-minute cell attachment period, while IC circulation rate was zero and the bioreactor was in the stationary mode. In steps 2, 4 and 6: the cell suspension in the IC circuit was circulated alternately in the positive and negative directions at sequentially lower circulation rates: −100 ml/min, 50 ml/min, and −25 ml/min for 2, 4 and 8 minutes. Once this task was completed, the system was put into the ‘Attach Cells’ stationary task mode, which allows the cells to adhere to the IC membrane surface. During this task, the IC media flow rate was interrupted (flow rate zero) to allow cell attachment, while the Extra-capillary (EC) flow rate was set at 30 mL/min to maintain gas exchange in the system. The cells were allowed to attach for 24 hours followed by a ‘Feeding’ step as described below.

### MSCs Feeding Programs

MSC P1 were propagated for six to seven days in PM with a feeding program starting with 48 hours at 0.1 ml/min IC inlet rate. Subsequently, the inlet rate was automatically doubled to 0.2 ml/min for 1 day and to 0.4 ml/min for one more day. Glucose and Lactate measurements (GlucCell, CESCO Bioengineering and Lactate Plus Lactate Meter, Nova Biomedical, respectively) were measured daily from the Sampling port (EC circulation loop). Each time the lactate concentration reached 4 mmol/L, the inlet rate was doubled up to a maximum rate of 1.6 ml/min. utilizing the Quantum ‘Feed Cells’ task with the fresh PM added to the IC compartment.

After 6-7 days in PM, differentiation was induced by replacing the PM with the differentiation media (S2M) and maintaining the cultures in S2M for three days. The differentiation stage was preceded by the ‘IC/EC Washout’ task with an exchange of 2.5 volumes of DMEM (IC volume 200 ml EC volume 300 ml). The purpose of this task was to wash the IC and the EC circulation loop to prepare the system for adding the S2M. Following this step, the S2M media bag was connected to the “IC media” line and the “Condition Media” task was started. The purpose of this task was to provide rapid contact between the media and the gas supply by using a high EC circulation rate (250 mL/min) while maintaining the IC circulation rate at 100 mL/min.

The S2M is a defined medium devoid of serum comprising DMEM, L-Glutamine, Sodium Pyruvate, di-butyryl cyclic AMP (dbcAMP), human Basic Fibroblast Growth Factor (bFGF), human platelet derived growth factor (PDGF-AA), human Heregulinβ, and supplemented with L-ascorbic acid 2-phosphate sesquimagnesium salt hydrate (Ascorbic acid, <1 kDa) for improving cell attachment. The main challenge in the transition from the proliferation to the differentiation stage is to ensure that it will not impair the high number of live cells in the bioreactor. To ensure cells remain adherent to the hollow fibers and are not washed-out during differentiation, the IC inlet rate was decreased, and S2M was added to the culture via continuous perfusion at 0.4 mL/min. This was accomplished by directing the IC circulation pump at half the flow rate (−0.2 mL/min) in the opposite (or negative) direction of the IC inlet rate at the same time. Without these opposing pump directions, even a small IC inlet rate can result in cells detaching from the bioreactor into the header space where a growing cell mass can accumulate.

Lowering the IC inlet rate from 1.6 mL/min to 0.4 mL/min leads to a decreased rate of glucose supply, therefore, to increase the supply of glucose to the cells, an M2 media bag was connected to the “EC media” line. M2 is the basic serum-free media composed of DMEM L-Glutamine, Sodium Pyruvate. M2 supplemented with Ascorbic acid was added to the EC inlet via continuous perfusion at 0.4 mL/min for 2 days that was increased to 0.8 mL/min on the third day of differentiation. The high number of MSCs reached within the IC circulation loop at the end of the propagation step requires abundant gas supply as well as lactate removal from the culture. This was accomplished by EC perfusion with M2 medium and by circulating the EC contents at a circulation rate of 300 mL/min.

The molecular weight cutoff of the Quantum system hollow fibers is approximately 17 kDa. The lower molecular weight of some of the components of the S2M differentiation medium, Heregulin B (7.5 kDa), cAMP (<1 kDa) may permit them to diffuse through the hollow-fiber membrane and dramatically reduce their effective concentration in the IC circulation loop. The molecular weight of Ascorbic acid (<1 kDa) would also allow it to pass through the semi-permeable membrane into the EC circulation loop. To overcome this challenge and maintain the balance Ascorbic acid was added to the EC medium.

To evaluate whether Ascorbic acid is essential for achieving a sufficient number of MSC-NTF cells in the Quantum System, two parallel systems were used for each tested donor: one system supplemented with Ascorbic acid in the medium and a second system, as a control, without Ascorbic acid in the medium.

The number of MSC-NTF cells harvested from the process in the Quantum System with Ascorbic acid (217±43×10⁶ cells) was significantly (p=0.05) higher as compared to the number of cells harvested from the Quantum System without Ascorbic acid (117±24×10⁶ (Table 2). Viability of harvested cells was an average of 95-96% for all processes (Table 2). The addition of Ascorbic acid significantly improved (p=0.003) the yield of MSC-NTF cells in the Quantum system. A reduction of oxygen concentration (O₂) in the gas mixtures from 20% to 5% increased the number of MSC-NTF cells harvested from the process in the Quantum System under hypoxic (5% O₂) conditions by 12% as compared to normoxic condition (288.86×10⁶ vs. 256.5×10⁶ cells). No significant difference between MSC-NTF cells in CD markers and Annexin V.

TABLE 2
MSC-NTF cells yield.

| Number of harvested MSC-NTF cells (×10⁶) | | | Viability (%) | |
|---|---|---|---|---|
| Donor | Quantum | Quantum with | Quantum | Quantum with |
| Number | (Control) | Ascorbic Acid | (Control) | Ascorbic Acid |
| D48 | 121 | 168 | 95 | 95 |
| D49 | 112 | 220 | 96 | 96 |
| D54* | 144 | 254 | 96 | 98 |
| D55 | 129 | 269 | 96 | 95 |
| D59 | 77 | 174 | 93 | 96 |
| Mean | 117 | 217 | 95 | 96 |
| SD | 24 | 43 | 1 | 1 |
| Maximum | 144 | 269 | 96 | 98 |
| Minimum | 77 | 168 | 93 | 95 |

| | | |
|---|---|---|
| t-Test | P = 0.05 | P = 0.66 |

*20 × 10⁶ P1 MSCs were seeded in each Quantum System with the exception of D54 where 10 × 10⁶ P1 MSCs cells were seeded.

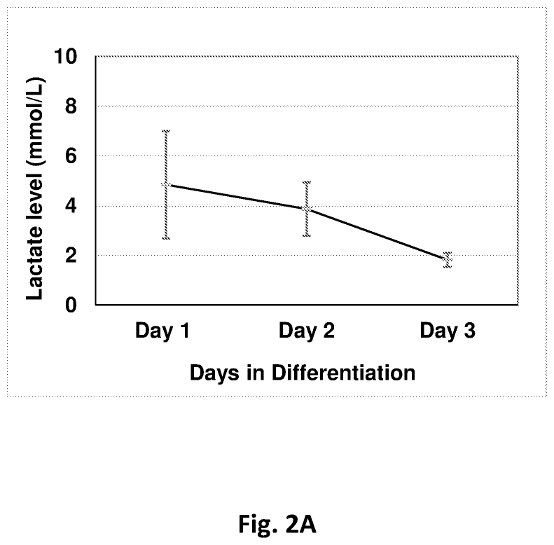

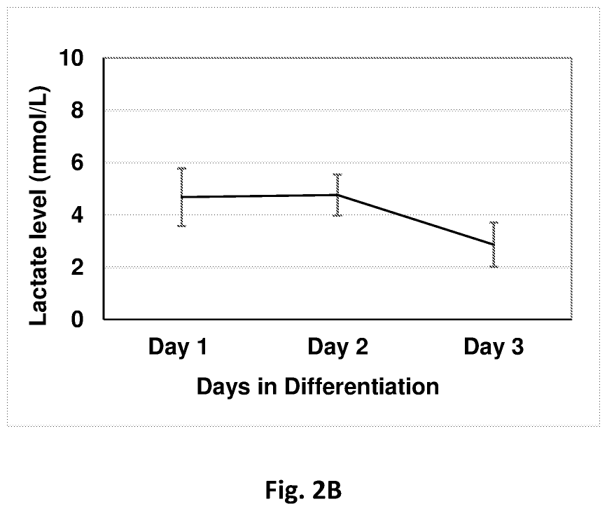

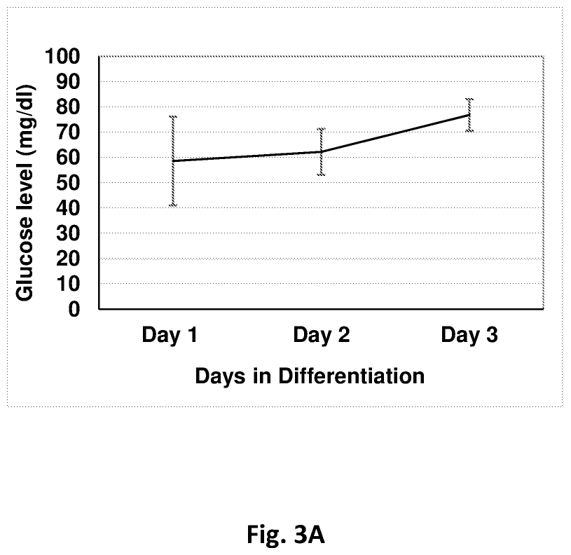

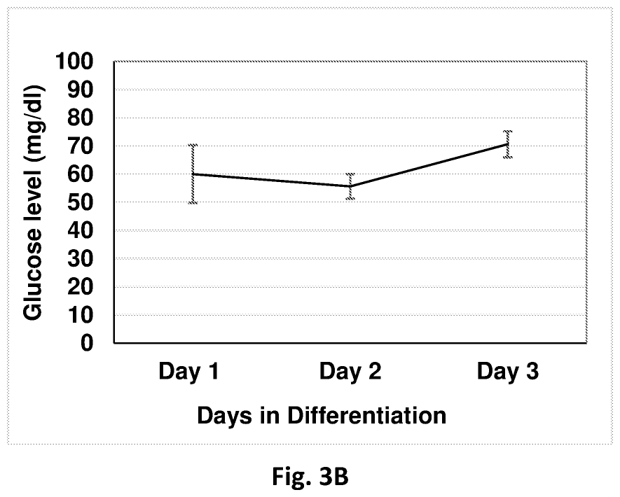

In the Quantum system Glucose and Lactate parameters were measured daily for monitoring the status of the culture. On the day of seeding, starting levels were 100-120 mg/dl of Glucose and 0-2 mmol/L of Lactate. Differentiation was induced when the cultures had exhausted their proliferation potential as determined by lactate levels above 4 mmol/L that could not be further reduced by increasing the flow rate to 1.6 mL/min. Before inducing differentiation, the Glucose and Lactate levels were 81 mg/dl and 6 mmol/L, respectively (data represent average of 10 runs with no correlation to the presence of Ascorbic acid in the medium). The metabolism rate was monitored during the MSC-NTF cells differentiation step. A trend of decrease in glucose consumption and lactate generation was observed suggesting a non-proliferating cell population (FIGS. 2A-2B and FIGS. 3A-3B). Addition of Ascorbic acid to the medium slightly, but not significantly increased those parameters (Glucose p=0.11, Lactate p=0.25 on the third day of differentiation, FIGS. 2A-2B and FIGS. 3A-3B), which correlated to the number of harvested cells (Table 2).

### Example 2—MSC-NTF Cells Characterization by Flow Cytometry

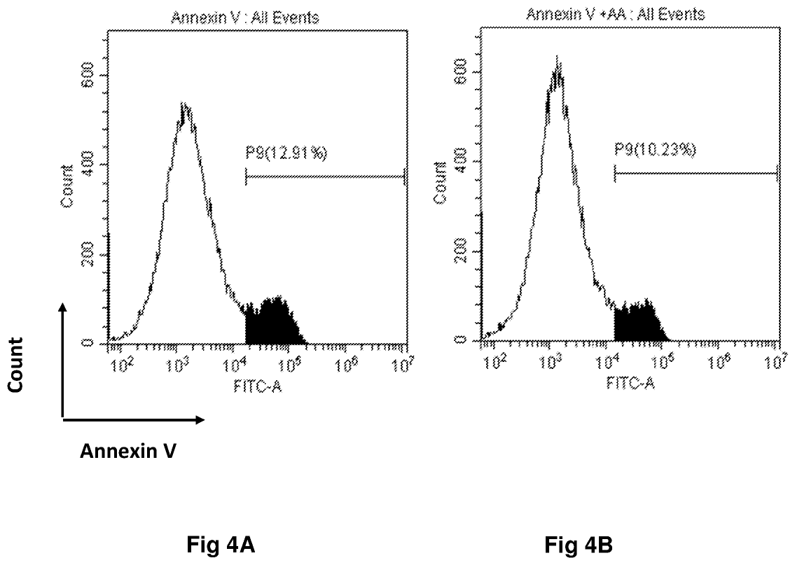

Fresh harvested MSC-NTF cells were analyzed for cell surface antigen expression and apoptosis by Flow cytometry. Apoptosis was measured using the Annexin V Apoptosis Detection Kit FITC (eBioSience). The acceptance criteria for MSC-NTF cells final product are expression of more than 90% CD73, CD90, CD105 surface markers and less than 20% Annexin V positive cells. The presence of Ascorbic acid in the medium did not affect the expression of CD markers or Annexin V positive MSC-NTF cells (CD73 p=0.32, CD90 p=0.25, CD105 P=0.1 and Annexin V p=0.11, Table 3 and FIGS. 4A-4B).

TABLE 3
Flow cytometry of MSC surface markers

| | Donor | | | | |
|---|---|---|---|---|---|
| Format | Number | CD73 | CD90 | CD105 | Annexin V |
| Quantum | D48 | 98 | 99 | 94 | 12 |
| system | D49 | 99 | 98 | 89 | 13 |
| (Control) | D54 | 99 | 99 | 92 | 16 |
| | D55 | 98 | 98 | 91 | 12 |
| | D59 | 88 | 91 | 84 | 23 |
| | Mean | 97 | 97 | 90 | 15 |
| | SD | 4 | 3 | 3 | 4 |
| | Maximum | 99 | 99 | 94 | 23 |
| | Minimum | 88 | 91 | 84 | 12 |
| Quantum | D48 | 99 | 99 | 94 | 10 |
| system | D49 | 99 | 98 | 91 | 10 |
| with | D54 | 99 | 99 | 96 | 9 |
| Ascorbic | D55 | 99 | 99 | 92 | 13 |
| Acid | D59 | 99 | 99 | 93 | 21 |
| | Mean | 99 | 99 | 93 | 12 |
| | SD | 0 | 0 | 2 | 4 |
| | Maximum | 99 | 99 | 96 | 21 |
| | Minimum | 99 | 98 | 91 | 9 |

| | | | | |
|---|---|---|---|---|
| t-Test | p = 0.32 | p = 0.25 | p = 0.10 | p = 0.11 |

### Example 3—VEGF Specific Productivity in MSC-NTF Cells

Molecular cloning reveals the existence of four species of vascular endothelial growth factor (VEGF) having 121, 165, 189, and 206 amino acids. These have strikingly different secretion patterns, which suggests multiple physiological roles for this family of polypeptides. The two shorter forms are efficiently secreted and are identified in the commercial ELISA assay, while the longer ones are mostly cell-associated (Ferrara N et al, 1991). On the day of harvesting, culture supernatants of MSC-NTF cells samples were collected from the Quantum system. In the Quantum system there is a continual replacement of medium. Therefore, at the end of the differentiation step VEGF was sampled twice, from the sample port (for testing media from the EC circulation loop, 300 ml) by syringe and from the waste bag (for testing media flow from both the IC and the EC circulation loops of the bioreactor 1-3 liter). The molecular weight cutoff for the hollow fiber in the Quantum system is approximately 17 kDa. It can be therefore assumed that part of the two VEGF shorter forms (VEGF121 and VEGF165, 18 and 23 kDa respectively) will diffuse through the membrane and be present in the EC circulation loop and the waste bag that collects both IC and EC media.

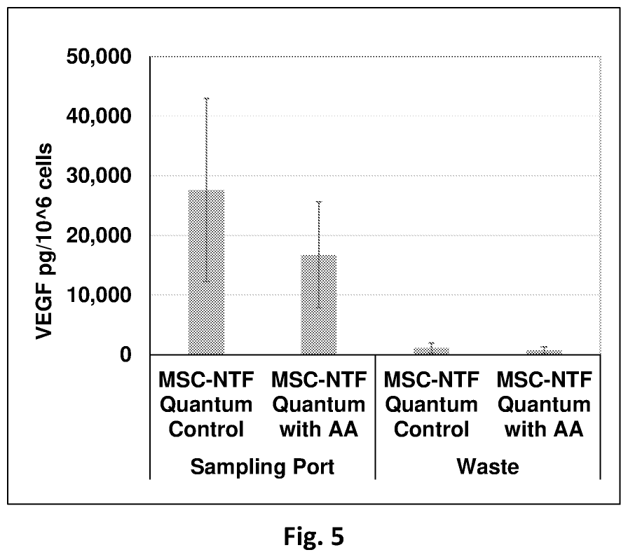

All samples were passed through a 0.22 μm filter, diluted 1:20 or 1:10 and used for VEGF detection by the Human VEGF Quantikine ELISA Kit (R&D systems). VEGF was measured at 450 nm in a Tecan microplate reader (Tecan, USA) and analyzed using the Magellan software. The specific productivity (VEGF secretion per 1×10⁶ MSC-NTF cells) was calculated based on the number of harvested cells (Table 2). VEGF specific productivity in the Quantum system sampled from the sampling port of the bioreactor supplemented with Ascorbic acid, was significantly lower as compared to the Quantum system control (FIG. 5. Sampling port: p=0.03, with AA 16,760±8,867 vs. without AA 27617±15354). VEGF specific productivity in the Quantum systems in the Waste bag was similar with or without Ascorbic acid (p=0.44, 738±601 vs. 1,099±877). The low levels in the Waste bag suggests that the majority of VEGF may accumulate in the IC circulation loop.

The acceptance criteria for MSC-NTF cells final product is >7000 pg/10⁶ cells. Despite the difference in VEGF secretion levels, it can be concluded that the potency of MSC-NTF cells processed in the Quantum system complies with the acceptance criteria. For additional confirmation, harvested MSC-NTF cells from the Quantum system were seeded in triplicate T-225 flasks in growth medium (PM), at a density of 10×103 cells/cm2 for three (3) days. Cells were then harvested; viable cell number were counted and culture supernatant collected for examining the cells' VEGF specific productivity by ELISA. VEGF specific productivity of MSC-NTF cells after 3 days in growth medium was significantly higher as compared to the sample collected form the Quantum system Sampling coil (for testing media from the IC circulation loop, 200 ml) (21,483 pg/10⁶ vs. 996 pg/10⁶ cells).

### Example 4—MSC-NTF Cells Gene Expression in Quantum

The MSC-NTF cells were characterized based on the pattern of expression of selected genes: BMP2, PSCK1, HGF and TOP2A shown to be modulated in MSC and MSC-NTF cells manufactured in the culture vessels. RNA from frozen MSC and MSC-NTF cells derived from the Quantum system were isolated using Quick RNA mini prep kit (Zymo research). cDNA was synthesized using the qScript cDNA synthesis kit (Quantabio) and Real-time qPCR performed using primer mix and SybrGreen for the following genes: BMP2, PSCK1, HGF and TOP2A, according to the list of primers in Table 4 below—

TABLE 4
List of primers for gene expression
analysis by RT-PCR

| Gene | | |
|---|---|---|
| name | Forward primer | Reverse primer |
| BMP2 | CCAGGTCCTTTGACCA | AAGCAGCAACGCTAGAAGACA |
| | GAGTT | |
| TOP2A | GTGGAGAAGCGGCTTG | ACCATCAAGCCTGCTCCATT |
| | GT | |
| PSCK1 | CCTGGAAGCAAACCCA | TTTCATTTACAGGCTGCAATGGT |
| | AATCTC | |
| HGF | CTTCAATAGCATGTCA | GCTGTGTTCGTGTGGTATCATG |
| | AGTGGAGT | |

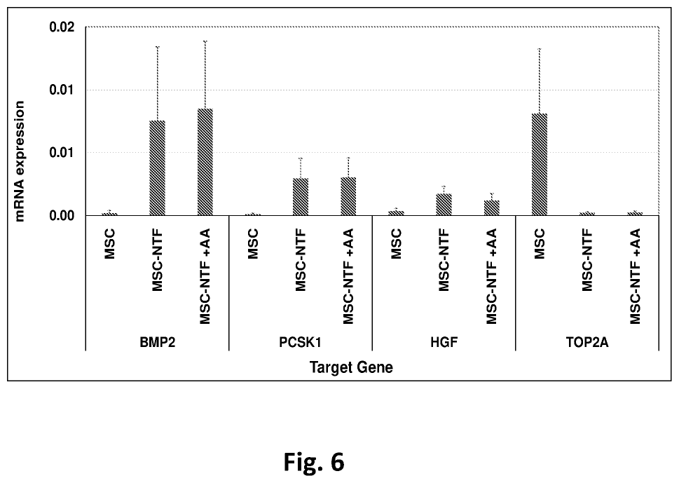

B2M and EF1A were used as the normalizing genes. The levels of gene expression were determined using the comparative Ct (Cycle threshold) method. A normalization factor calculated as the geometric mean of the quantity of the two normalizing genes (B2M and EF1A) was used to normalize the expression levels for each gene. Since there is a high variability between donors, we selected genes that can or cannot be expressed unambiguously in MSCs, and the selected genes do not necessarily have a biological function. The expected pattern of gene expression in MSCs is lack of expression of BMP2, PCSK1 and HGF genes, and expression of TOP2A. When MSCs differentiated into MSC-NTF cells the expression of TOP2A is downregulated and the other genes are upregulated, such that MSC-NTF cells differentiation leads to a “mirror picture” of MSC genes expression. The gene expression of MSC-NTF cells produced in the Quantum system with or without Ascorbic acid was similar with no significant difference (BMP2 p=0.34, PSCK1 p=0.63, HGF p=0.24 and TOP2A p=0.37, FIG. 6) and were significantly different from MSCs expanded in Quantum (BMP2 p=0.01, PSCK1 p=0.004, HGF p=0.02 and TOP2A p=0.01, FIG. 5).

### Example 5—MSC-NTF Cells Immunomodulation Assay

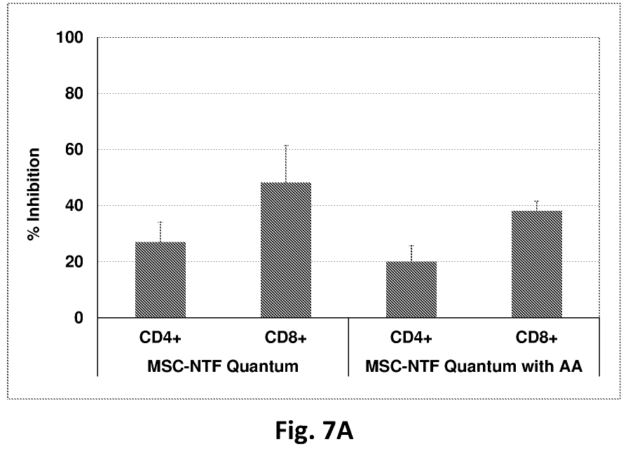

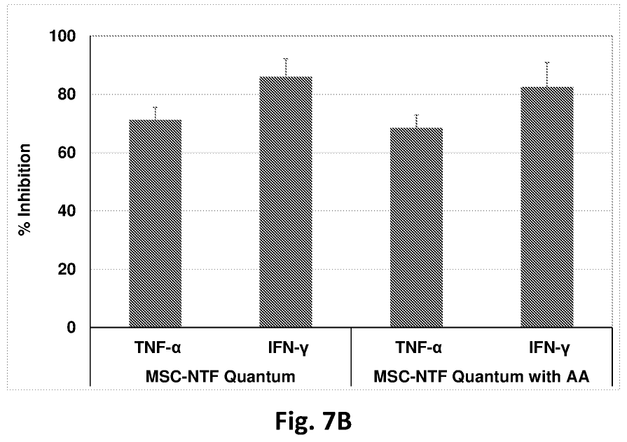

To confirm their immunomodulatory properties, MSC-NTF cells were co-cultured with CFSE-labeled, activated PBMC. CFSE can bind irreversibly to intracellular and cell-surface proteins and is subsequently distributed equally between daughter cells upon cell division. As a result, halving of cellular fluorescence intensity marks each successive generation in a population of proliferating cells and can be readily followed by flow cytometry. After 4 days of co-culturing, the PBMC and supernatants were collected. CFSE-labeled PBMC were used to track the proliferation of CD4+ and CD8+ T-cells by FACS analysis and the cell culture supernatants were used for measuring the secretion of TNF-α and IFN-γ by ELISA assays using Human DuoSet ELISA kits (FIGS. 7A-7B). The percentage of proliferating CD4+ and CD8+ T cells was measured by flow cytometry and was calculated as a percentage of the activated PBMC cells, which were cultured alone as a positive control. TNF-α and IFN-γ secretion was measured in the supernatants of non-activated and activated PBMCs cultured in the presence of MSC-NTF cells. Inhibition of cytokines secretion was calculated as a percentage of the activated PBMC control. No significant difference was observed in the ability to inhibit T cells, which is associated with decrease in both TNF-α and IFN-γ in culture supernatants, of MSC-NTF cells produced by the Quantum system with or without Ascorbic acid (p=0.36 and p=0.52, respectively, FIGS. 7A-7B). MSC-NTF cells produced by the Quantum system are capable to inhibit the secretion of TNF-α and IFN-γ by 68% and 83%, respectively.

### Example 6—MSC-NTF Cells Induce Neurite Extension

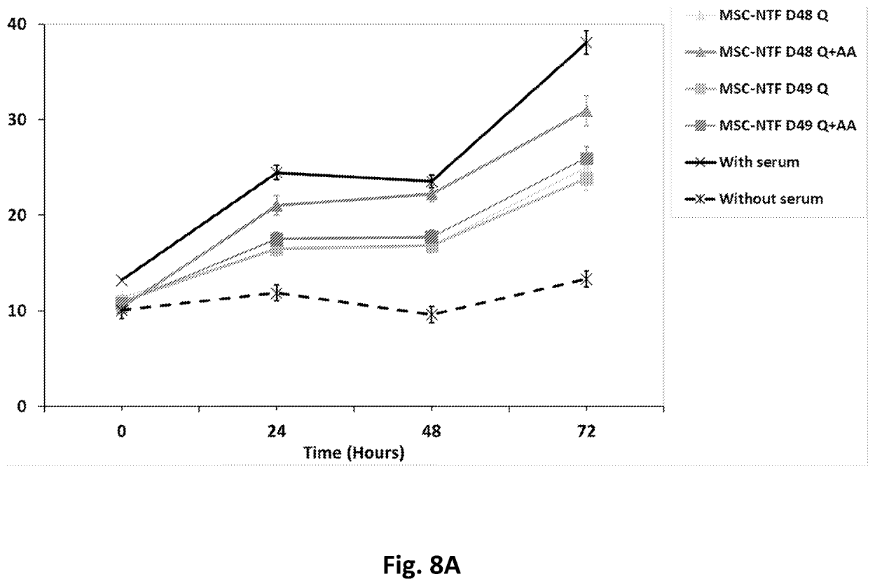

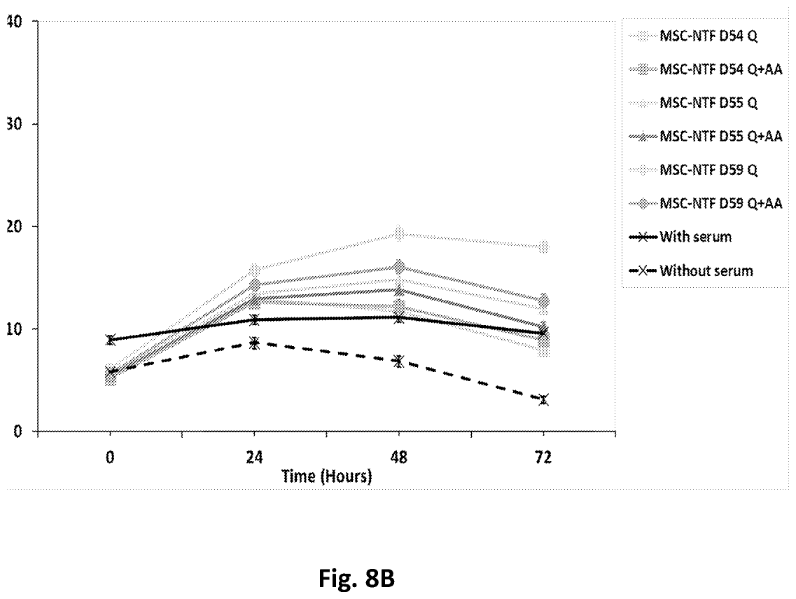

MSC-NTF cells induces neurite outgrowth when co-cultured with the SH-SY5Y neuronal cell line, suggesting an enhanced functional response of neuronal cells to MSC-NTF cells. MSC-NTF cells secrete functional NTFs that impact neuroprotective mechanisms and, therefore, hold therapeutic potential. MSC-NTF cells produced in the Quantum system (±AA) induced variable neurites outgrowth as compared to control samples, SH-SY5Y neuronal cell line without serum (dotted line) in the medium. (FIGS. 8A-8B).

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A method for generating cells which secrete neurotrophic factors (NTFs) in a functionally closed and automated hollow-fiber bioreactor system comprising inducing differentiation of a population of undifferentiated mesenchymal stem cells (MSCs) in a differentiating medium supplemented with ascorbic acid.

2. The method according to claim 1, wherein the yield of said cells is improved in comparison to generation said cells in a differentiation medium without ascorbic acid.

3. The method according to claim 1, wherein said ascorbic acid concentration in the differentiating medium is 250 μM.

4. The method according to claim 1, further comprising culturing said population of undifferentiated mesenchymal stem cells (MSCs) prior to said induction of differentiation, wherein said culturing is affected under conditions that do not promote cell differentiation.

5. The method according to claim 4, wherein said population of undifferentiated mesenchymal stem cells (MSCs) is cultured in a functionally closed and automated hollow-fiber bioreactor system.

6. The method according to claim 4, wherein said culturing duration is based on lactate parameters.

7. The method according to claim 1, wherein said differentiating medium is designated S2M media.

8. (canceled)

9. The method according to claim 1, further comprising M2 media comprising DMEM L-Glutamine and Sodium Pyruvate supplemented with Ascorbic Acid.

10. The method according to claim 1, comprising—
a. Seeding between 10-20×10⁶ MSC in growth media (PM) for six or seven days;
b. Propagating MSC by using a feeding program based on lactate level measurements;
c. Replacing the PM with the differentiating media (S2M) six or seven days after seeding;
d. Incubating the cultures for three additional days;
e. Harvesting the MSC-NTF cells.

11. The method according to claim 1, further comprising analyzing the expression of CD73, CD90 and CD105 surface markers.

12. (canceled)

13. (canceled)

14. (canceled)

15. The method according to claim 1, further comprising analyzing VEGF specific productivity.

16. An isolated population of cells, which secrete neurotrophic factors, generated according to the method of claim 1.

17. (canceled)

18. (canceled)

19. The isolated population of cells according to claim 16, wherein said cells secrete not less than 7000 pg VEGF/10⁶ cells.

20. A method of treating a disease for which administration of neurotrophic factors is beneficial in a subject in need thereof, comprising administering to the subject a therapeutically effective amount of the isolated population of cells according to claim 16, thereby treating said disease.

21. The method of claim 20, wherein said cells are ex vivo differentiated from MSCs which are autologous, or allogenic to said subject.

22. (canceled)

23. (canceled)

24. The method of claim 20, wherein said disease is a neurodegenerative disease or an immune disease.

25. The method of claim 24, wherein said neurodegenerative disease is selected from the group consisting of Parkinson's, Multiple System Atrophy (MSA), multiple sclerosis, epilepsy, amyotrophic lateral sclerosis (ALS), stroke, autoimmune encephalomyelitis, diabetic neuropathy, glaucomatous neuropathy, Alzheimer's disease, and Huntington's disease.

26. (canceled)

27. (canceled)

28. (canceled)

29. The method of claim 20, wherein said administration is intramuscularly or intrathecally.

30. The method of claim 29, wherein when said administering is intramuscularly, a total amount of cells administered to a subject is between 20-100×10⁶ cells per administration, wherein when said administering is intrathecally, an amount of MSC-NTFs administered to a subject is between 100-125×10⁶ cells per administration, and wherein when said administering is intrathecally and intramuscularly, a total amount of MSC-NTFs administered to a subject is between 20-500×10⁶ cells.

31. (canceled)

32. (canceled)

33. The method according to claim 20, wherein said administration is a single administration, or wherein said administration is a repeated administration of up to 10 administrations.

34. (canceled)

35. (canceled)

36. (canceled)
